(19) United States
(12) Patent Application Publication
(10) Pub. No.: US 2026/0259140 A1
(43) Pub. Date: Sep. 3, 2026

(54) METHOD OF EVALUATING THE FUNCTIONALITY OF EXTRACELLULAR VESICLE PREPARATIONS

(71) Applicant: CONSIGLIO NAZIONALE DELLE RICERCHE, Roma (IT)

(72) Inventors: Antonella BONGIOVANNI, Palermo (IT); Giorgia ADAMO, Palermo (IT); Sabrina PICCIOTTO, Marsala (Trapani) (IT)

(21) Appl. No.: 19/163,723

(22) PCT Filed: Mar. 7, 2024
(86) PCT No.: PCT/IB2024/052194

§ 371 Date: Sep. 9, 2025

(30) Foreign Application Priority Data
Mar. 10, 2023 (IT) 102023000004503

(51) Int. Cl. G01N 21/64

(52) U.S. Cl. CPC G01N 21/6408; G01N 21/6428; G01N 2021/6439; G01N 2333/916

(57) ABSTRACT

A method of evaluating the functionality of a preparation of extracellular vesicles (EVs) involves incubating the preparation of EVs with fluorescein diacetate (FDA) as a non-fluorescent precursor of fluorescein under suitable conditions so that the FDA enters the EVs of the preparation and is hydrolysed by endogenous esterase enzymes, thereby forming fluorescence-emitting fluorescein molecules. The amount of fluorescein produced, being related to specific intravesicular enzymatic activity, provides a quantitative measure of the functionality of the preparation of EVs.

The present invention refers to a method of evaluating the functionality of a preparation of extracellular vesicles (EVs), in particular for assessing the quality and biological activity of the preparation, in order to validate its use for therapeutic, pharmaceutical and/or cosmetic applications.

In order to be able to translate the application of extracellular vesicles, including exosomes, from basic science to the clinic or cosmeceutics, it is essential that the quality of the EV preparations is reproducible and meets established quantitative requirements and criteria for efficacy, before these products are used in clinical trials. In fact, the composition of the EV preparations is known to largely depend on the originating cell system and the processes by which the vesicles were isolated. Therefore, it is of great importance to optimize the production of the extracellular vesicle preparations while developing criteria allowing the identity and biological activity of the vesicles to be established, for example through specific tests, prior to their application as new drugs for clinical trials, therapies, or as cosmeceutical products.

To date, a wide range of in vitro and in vivo assays has been used to study the identity, quality, and function of extracellular vesicles. In this context, specific assays suitable for evaluating the bioactivity of EVs include biochemical analyses, or tests based on the use of animals, organoids, tissues, and cells. However, several limitations make it difficult to set up and develop reliable assays to quantitatively assess the bioactivity of EVs. In fact, many authors have to face the problem of the lack of tests that allow preliminary assessment of the biological potency of an EV preparation, where the term “potency” is defined by the US Food and Drug Administration (FDA) in its “Guidance for Industry 2011: Potency Tests for Cellular and Gene Therapy Products” as “the specific ability or capacity of the product, as indicated by appropriate laboratory tests or by adequately controlled clinical data obtained through the administration of the product in the manner intended, to effect a given result” (https://www.fda.gov/media/79856/download). In addition, according to the guidelines of the International Council for the Harmonization of technical requirements for pharmaceuticals for human use (ICH), in the context of technical procedures and approval criteria for biotechnological/biological products, a potency assay is a quantitative biological assay that measures the biological activity of a product, which is linked to certain intrinsic biological properties (https://database.ich.org/sites/default/files/Q6B_Guideline).

Typically, potency assays described for EVs are “disease-specific”. For the development of such assays, it is therefore necessary to initially determine the pathological processes that can be modulated by EVs in a preclinical animal model (Nguyen, V V, et al. “Functional assays to assess the therapeutic potential of extracellular vesicles”. J Extracell Vesicles. 2020; 10:e12033. https://doi.org/10.1002/jev2.12033).

The studies described in Bruno et al. (Bruno S, et al. “Renal Regenerative Potential of Different Extracellular Vesicle Populations Derived from Bone Marrow Mesenchymal Stromal Cells”; Tissue Eng Part A. 2017; 23(21-22):1262-1273) indicated that EV preparations derived from mesenchymal stem cells (MSC), ranging in size from approximately 80 nm to 1 μm, exert a protective activity against glycerol-induced acute kidney injury. The potency tests developed by the authors to predict the potency of such EV preparations are based on quantitative analyses carried out using real-time PCR aimed at measuring the levels of specific transcripts in the vesicles. This approach, however, is only linked to one type of specific and sectoral application.

On the other hand, assays for the evaluation of the potency of an EV preparation that are universal and specific for any type of EV should reflect their Mechanism Of Action (“MOA”), but this is challenging not only because extracellular vesicles are very heterogeneous but also because they have different functional activities (such as, for example, anti-oxidant, anti- or pro-inflammatory, neuro-regenerative, epithelial-regenerative activities, etc.). Therefore, to test the potency of EVs isolated from various biological sources and for different therapeutic applications with a single disease-specific test, it is necessary to develop a specific assay which is validated ad hoc for each therapeutic use.

In recent years, experimental protocols have been described, which use enzyme substrates permeable to the phospholipid envelope of vesicles to make them fluorescent, including carboxyfluorescein diacetate-acetoxymethyl ester (CFDA), carboxyfluorescein diacetate succinimidyl ester (CFSE) and calcein acetoxymethyl ester (calcein-AM) (Gray W D, et al. “An accurate, precise method for general labeling of extracellular vesicles”, MethodsX. 2015; 2:360-367; Ender F, et al., “Detection and Quantification of Extracellular Vesicles via FACS: Membrane Labeling Matters!”, Int J Mol Sci. 2019; 21(1):291). However, the use of these substrates is restricted to the sole purpose of making the EVs fluorescent in order to be able to identify and quantify them by flow cytometry analysis (nanoFACs). Indeed, these methods are not quantitative and do not provide any information regarding the functional activity and quality of EV preparations.

Therefore, there is a need to provide a method for accurately, quantitatively and reproducibly determining the quality and bioactivity of a preparation of extracellular vesicles, which is suitable for use on vesicles of different types, regardless of their origin and/or of the method of preparation of these vesicles, thus resulting in wide application range.

This and other needs are met by the method as defined in the appended claim 1.

Additional features of the invention are identified in the dependent claims, which, together with the independent claims, form an integral part of the specification.

Within the scope of the present description, the term “functionality of a preparation of extracellular vesicles” refers to a general quantitative measurement of the intravescicular enzyme activity, which is representative of the biological activity of the EVs and at the same time indicative of the integrity of the lipid bilayer membrane that forms the vesicle envelope.

In other words, within the scope of the present description, the term “functionality” refers to a measurement of the general biological activity of the extracellular vesicles of a preparation (measured as an enzymatic activity), which is also related to their structural integrity.

The term “potency assay”, as used herein, refers to a method for the determination in an EV preparation of a particular biological activity, generally associated with a specific disease, the application of which is therefore limited only to certain therapeutic or diagnostic areas.

As will be explained in greater detail below, the method according to the invention advantageously enables an overall assessment of the quality and bioactivity of the test EV preparation, independent of the type or origin of the vesicles, which is therefore an essential prerequisite for a subsequent therapeutic use of this preparation, and therefore for the relevant potency assay. In fact, where the result of the method of the invention indicates no or little bioactivity of an EV preparation, that preparation is unsuitable a priori for therapeutic use, thus avoiding the need to carry out a subsequent specific, laborious and costly potency assay.

The method of the present invention is based on the use of fluorescein diacetate (3′-6′-diacetyl-fluorescein; FDA) as a substrate to measure the activity of enzymes, such as esterases, present in extracellular vesicles. As is well known in the art, FDA is a fluorogenic ester which, as a result of the enzymatic hydrolysis of the two acetate radicals, is cleaved to form fluorescein.

The present inventors surprisingly found that esterase enzymes are part of the biocargo of a large number of extracellular vesicles, which can also be very different from each other, such as, for example, vesicles derived from human cells or body fluids, plant vesicles, and vesicles derived from microalgae (Pocsfalvi G, et al, “Protein biocargo of citrus fruit-derived vesicles reveals heterogeneous transport and extracellular vesicle populations”, Journal of Plant Physiology 2018; 229:111-121; Gonzalez-Begne M, et al, “Proteomic analysis of human parotid gland exosomes by multidimensional protein identification technology (MudPIT)”, J Proteome Res. 2009 March; 8(3):1304-14 doi: 10.1021/pr800658c. PMID: 19199708; PMCID: PMC2693447; Gonzales P A, et al “Large-scale proteomics and phosphoproteomics of urinary exosomes”, J Am Soc Nephrol. 2009 February; 20(2):363-79. doi: 10.1681/ASN.2008040406. Epub 2008 Dec. 3. PMID: 19056867; PMCID: PMC2637050) and surprisingly noted that there was a correlation between the magnitude of the intravescicular esterase activity and the integrity and bioactivity of the vesicles. Therefore, the activity of esterase enzymes advantageously represents a common measurement for EV preparations which is indicative of the quality and biological activity thereof, regardless of the origin and biogenesis of the vesicles.

The method according to the invention comprises an initial step of incubating an EV preparation with non-fluorescent fluorescein diacetate (FDA) under suitable conditions so that fluorescein diacetate enters the EVs of the preparation by crossing the lipid bilayer membrane. Inside the vesicles, fluorescein diacetate is hydrolysed by specific enzymes, such as endogenous esterases, thereby forming fluorescence-emitting fluorescein molecules, which, as is well known, are negatively charged and impermeable to lipid membranes.

In one embodiment according to the invention, fluorescein diacetate is added to the preparation of EVs at a concentration comprised within the range of 0.0005% to 0.001% w/v based on the total volume of the preparation of EVs, more preferably at a concentration of 0.00075% w/v based on the total volume of the preparation of EVs.

In the method of the present invention, during the hydrolysis reaction by the esterase enzymes, the fluorescence intensity (expressed in arbitrary units, AU) emitted at a wavelength of 516 nm is detected over time, and the value of this fluorescence intensity is measured at a given time t of the hydrolysis reaction (Iₜ).

According to the invention, the intensity of the fluorescence emitted at the wavelength of 516 nm is detected by spectroscopy, preferably using a spectrofluorimeter.

The selection of the suitable instrument for detecting the fluorescence intensity in the method according to the invention falls well within the skills of those of ordinary skill in the art.

The fluorescence intensity emission in the method according to the invention occurs at a temperature between 21° C. and 25° C. (room temperature). Most preferred is a temperature of 22° C.

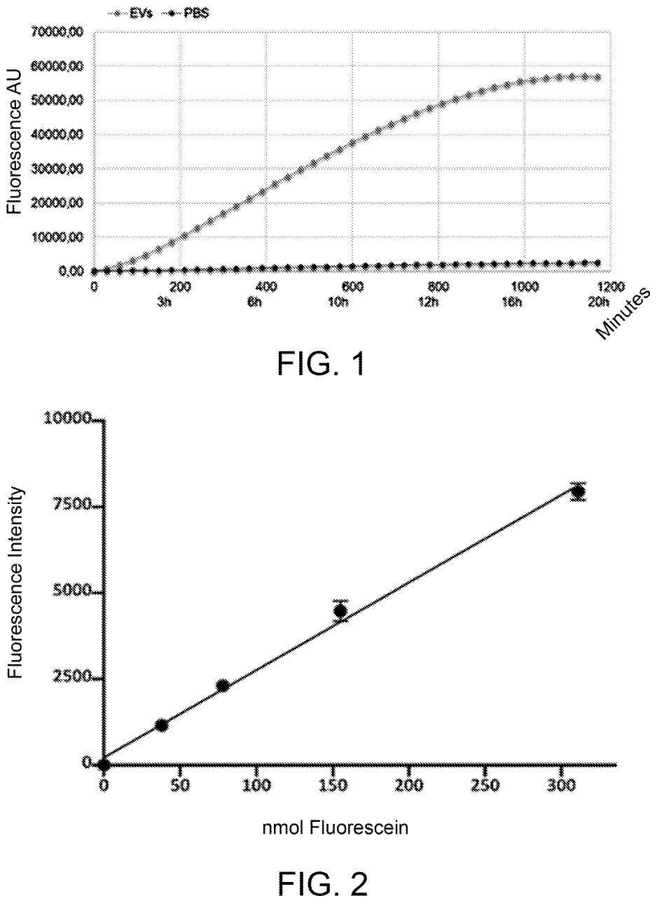

As will be explained in greater detail below, the fluorescent signal produced during the hydrolysis reaction of the method according to the invention has a curve that is a function of time and is represented by the graph shown in FIG. 1.

In a preferred embodiment of the invention, the value of the emitted fluorescence intensity is measured at a time t of the hydrolysis reaction in the phase of exponential increase in the fluorescence intensity.

In a preferred embodiment, the time t is between 60 and 600 minutes, e.g. 60, 90, 120, 150, 180, 210, 240, 270, 300, 330, 360, 390, 420, 450, 480, 510, 540, 570 or 600 minutes.

In a more preferred embodiment, the time t is between 120 and 240 minutes, more preferably 180 minutes.

According to the invention, the method comprises the further steps of:
determining the amount of fluorescein produced at time t in the preparation of EVs (Fₜ), using a fluorescein calibration curve in which the intensity of the fluorescence emitted at the wavelength of 516 nm is a linear function of the amount of fluorescein, preferably expressed in nmol, and
calculating from the above value Fₜ the amount of fluorescein produced per unit of time, preferably expressed in nmol/min, in the preparation of EVs (F) by applying the formula:

$$F = \frac{Ft}{t}$$

In the method according to the invention, fluorescein represents the equivalent reaction product released following the cleavage of FDA by the esterase enzymes present in the EVs.

The internationally used time measurement unit which is particularly preferred in the method of the invention is the minute.

It follows that the amount of fluorescein produced per unit of time in an EV preparation, for example expressed as nanomoles per minute, is related to the measurement of the specific enzymatic activity of intravesicular esterases, typically internationally expressed as an enzymatic unit (U=nmol/min). In this way, the final value returned by the method of the invention is a quantitative measure of the enzymatic activity of the EV preparation, i.e., a measure indicative of the vesicles' bioactivity and membrane integrity parameters.

In one embodiment of the method according to the present invention, the fluorescence intensity measured in the EV preparations per unit of time can be quantitatively defined as an enzymatic activity (U=nmol/min) using a calibration curve prepared with serial dilutions of fluorescein, preferably fluorescein sodium salt. The interpolation on the calibration curve of the fluorescence intensity measured in the EV preparations allows the amount of fluorescein produced in the preparation per unit of time, and thus the enzymatic activity of the EVs, to be obtained.

Extracellular vesicles are produced by many different cell types—the so-called donor cells—and are ubiquitously present in biological fluids and in cell or tissue cultures. According to the present invention, the method can be applied to determine the enzymatic activity of EV preparations obtained from different cell, animal and plant types, such as stem cells, normal and cancerous human cells, bacterial cells (Journal of Proteomics, Volume 231, 2021, 103994, ISSN 1874-3919), and cells from microalgae, and also from various biological fluids.

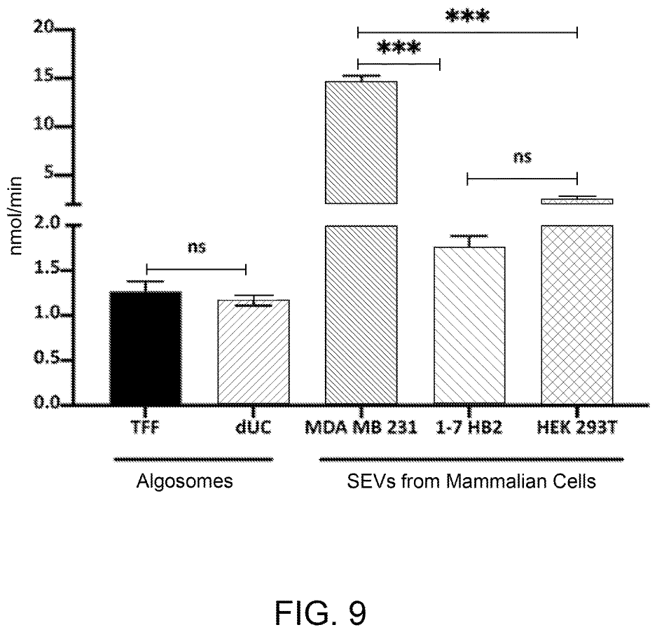

As shown in FIG. 9, the method of the present invention is suitable for use on preparations of EVs from human cell sources, e.g., extracellular vesicles derived from normal or tumour human cell lines.

According to one embodiment of the invention, the extracellular vesicles are derived from a biological fluid or from a conditioned cell or tissue culture medium.

As is known, a conditioned culture medium is a used medium collected from cell or tissue cultures. It contains metabolites, growth factors, and extracellular matrix proteins, which have been secreted into the medium by the cultured cells.

In a preferred embodiment, the extracellular vesicles are derived from a biological fluid selected from blood, saliva, plasma, serum, urine, or cerebrospinal fluid.

Methods suitable for achieving the isolation of extracellular vesicles from biological fluids, cell cultures, or tissue supernatants are known and described in the state of the art, therefore the selection and use thereof are well within the skills of those of ordinary skill in the art. Examples include, but are not limited to, density gradient, ultracentrifugation, filtration, dialysis, and capillary electrophoresis.

As will be explained in greater detail in the following experimental part, in order to validate the application of the method according to the invention, the present inventors carried out an accurate evaluation of the performance of said method.

The analytical sensitivity was first verified by determining the minimum amount of extracellular vesicles in the preparation that allows the production of fluorescein from the FDA substrate per unit of time to be detected, compared to the background. A correlation between the number of EVs in the test preparation and the amount of fluorescein measured per unit of time was found by using the method of the invention with a model of extracellular vesicles derived from microalgae ((nano)algosomes), with a detection limit of 6.25×10⁹ EVs/mL or greater (as shown in FIG. 4).

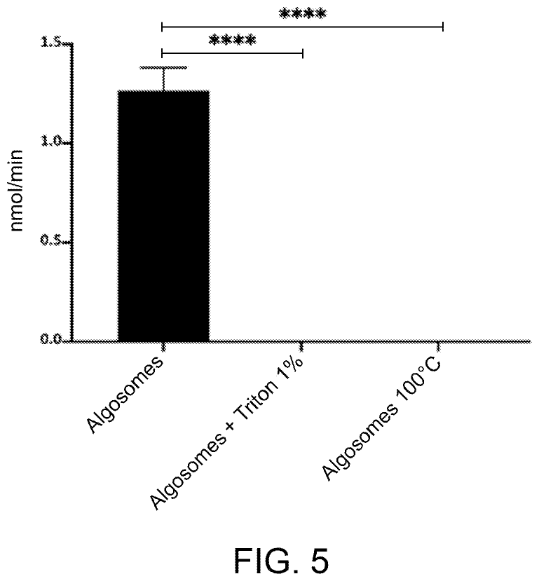

It has also been verified that the method according to the invention allows a measurement of the integrity of the vesicles, thus demonstrating that fluorescein was not produced from FDA when the EV preparations used in the above method had previously undergone a treatment for the lysis of the phospholipid membranes, for example, a detergent or boiling treatment (as shown in FIG. 5).

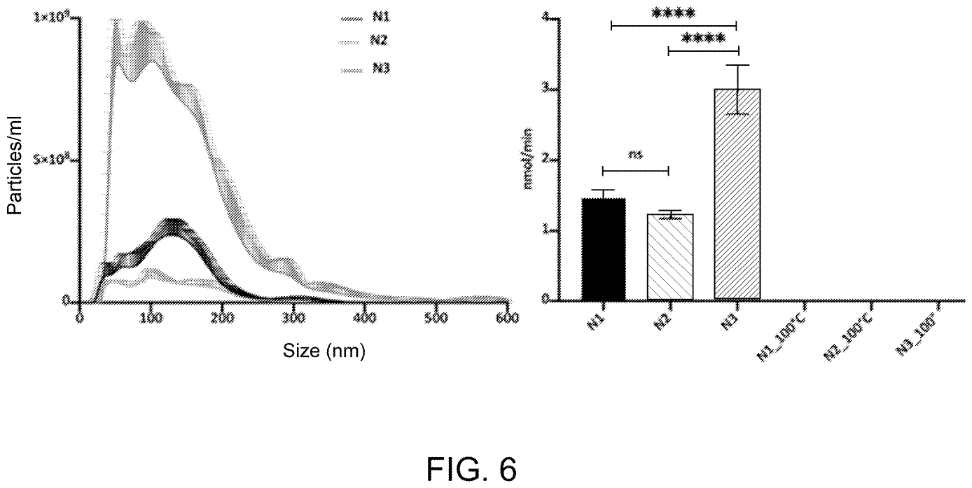

To further assess the performance of the method according to the invention, in particular the ability to provide a measure of the functionality of an EV preparation which is representative of the quality of said preparation, batches of (nano)algosome preparations, which differed from each other on the basis of parameters recognized by those of ordinary skill in the art as EV quality indicators, such as, for example, the size distribution of vesicles positive for a plasma membrane fluorescent marker (measured by fluorescence nanoparticle tracking), as well as the protein content thereof, were analysed. The graph in FIG. 6 shows that the magnitude of the enzymatic activity of the tested EV preparations, measured with the method of the invention and expressed as the amount of fluorescein produced per unit of time, correlates significantly with the quality parameters measured for the above preparations, thereby confirming the suitability of said method as a tool for validating EV preparations and monitoring their variability.

By virtue of the above, the method according to the invention therefore allows an accurate quantitative indication of the functionality of an EV preparation to be obtained in a short time and with a limited number of steps, which is not affected by the nature of the vesicles or how they were isolated, thus representing a universally quantifiable and standardizable value. This advantageously allows the method according to the invention to be used to perform qualitative evaluations on a given EV preparation, for example to predict in a medical context whether said preparation has the potential to achieve the expected therapeutic effects, or to make reliable comparisons between different preparations of vesicles, which can also be very heterogeneous.

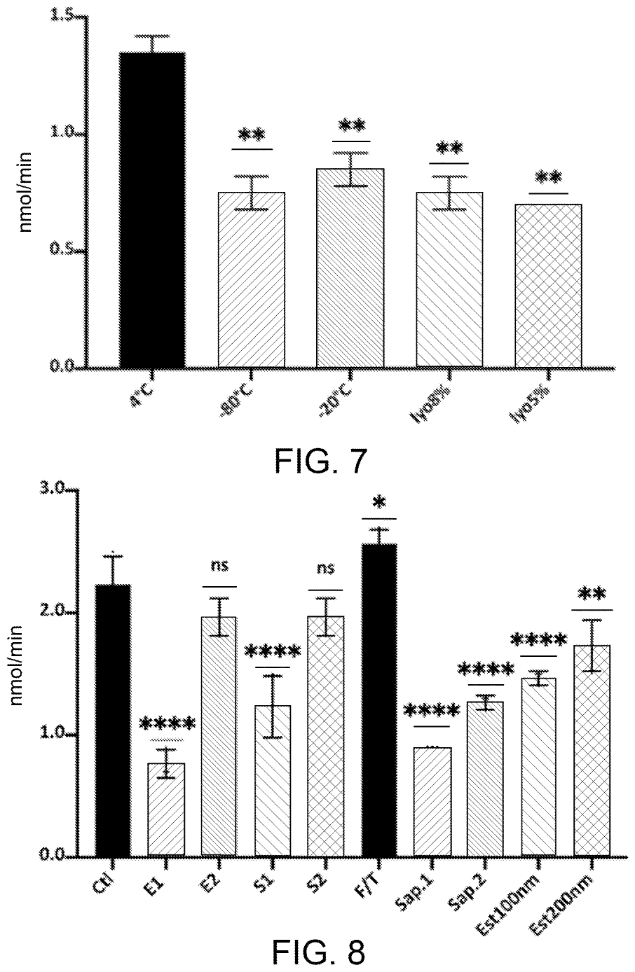

As shown in the experimental examples below, the method of the invention can be applied in different fields, particularly in the diagnostic and therapeutic field, and can be used, for example, to determine and compare bioactivity measurements of EV preparations that differ from each other as to the storage conditions (FIG. 7), the ways in which the vesicles were isolated (FIG. 8) and/or the procedures by which they were loaded with different molecules (FIG. 9).

Therefore, unlike the potency assays, which are aimed at measuring particular biological activities of EVs associated with applications in specific therapeutic areas, the present invention provides a method that allows a general assessment of the functional activity of the vesicles, by providing a measurement that is independent of the type and/or origin of the EVs in a preparation.

The experimental section that follows is provided for illustration purposes only and does not limit the scope of the invention as defined in the appended claims. In the following experimental section, reference is made to the attached drawings, wherein:

FIG. 1 shows a curve representing the time course of the fluorescence emission intensity in EVs incubated with the FDA substrate;

FIG. 2 shows a calibration curve for fluorescein sodium salt;

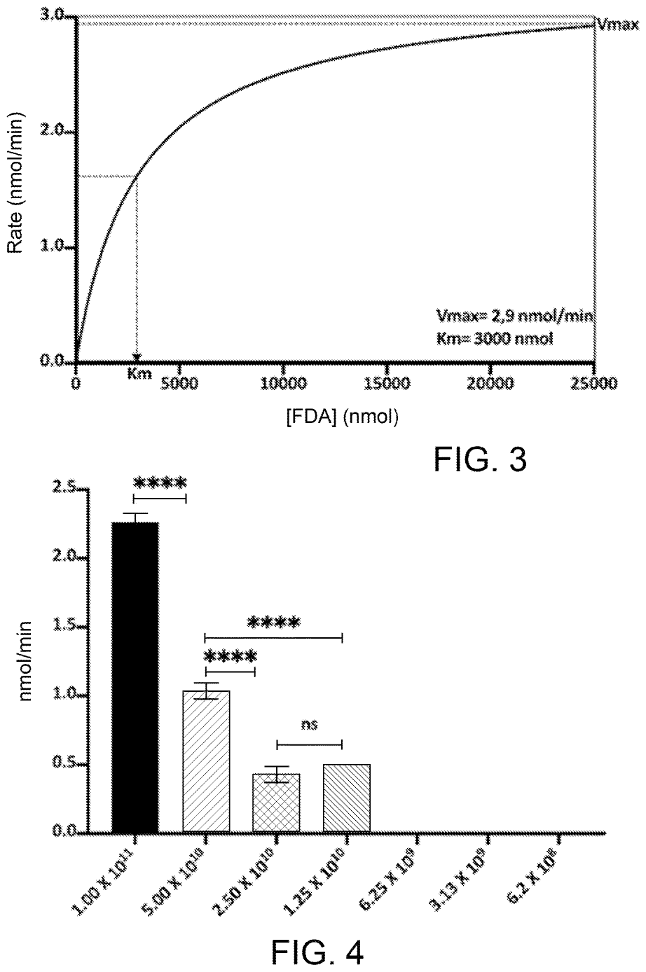

FIG. 3 shows a curve representing the enzymatic kinetics of the hydrolysis reaction of fluorescein diacetate (FDA) according to the Michaelis-Menten model;

FIG. 4 shows a graph illustrating the results of the method of the invention carried out on different concentrations of EVs;

FIG. 5 shows a graph illustrating the results of the method of the invention carried out on EV preparations subjected to lysis by heating at 100° C. or to treatment with 1% Triton X100;

FIG. 6 shows that the method according to the invention allows the quality of EV preparations to be assessed. Analysis by fluorescence nanoparticle tracking (left) and functionality assay results (right) on 3 different preparations of extracellular vesicles;

FIG. 7 shows the results of the method of the invention carried out on EV preparations after storage under different conditions (4° C., −20° C., −80° C., freeze-drying in 5% and 8% sucrose);

FIG. 8 shows the results of the method of the invention carried out on EV preparations in which the vesicles were subjected to different physical loading methods;

FIG. 9 shows the results of the method of the invention performed on preparations of EVs isolated by Tangential Flow Fractionation (TFF) and differential UltraCentrifugation (dUC) (first two bars of the histogram on the left) and on extracellular vesicles isolated from human, tumour and normal cells (last three bars of the histogram on the right).

### DECLARATION PURSUANT TO ART. 170 BIS OF THE CODE OF INDUSTRIAL PROPERTY

The present invention has been made in accordance with the provisions established by Article 170-bis, paragraphs 2, 3 and 4, of the Code of Industrial Property (Legislative Decree No 30, of 10 Feb. 2005, as amended by Legislative Decree No 131 of 13 Aug. 2010).

### Materials and Methods

### Set-Up of the Enzymatic Assay

### Preparation of the Fluorescein Standard Curve

Fluorescein sodium salt (Sigma-Aldrich) was used to generate a standard calibration curve for fluorescein (FIG. 2). The fluorescence intensity of fluorescein (serial dilutions from 300 nmol to 0 nmol in PBS without Ca⁺⁺ and Mg⁺⁺ at the final volume of 200 μl), was measured using a spectrofluorimeter (ex/em 490/514 nm, GloMax Discover Microplate Reader, Promega).

### Analytical Sensitivity

Serial dilutions of vesicles (1×10¹¹ to 6.2×10⁸ EVs/mL) were used to test the analytical sensitivity of the assay. The minimum/optimum concentration of vesicles (5×10¹⁰ vesicles/mL) to be used for the assay was selected based on the results obtained.

### Experimental Set-Up

Fluorescein diacetate (FDA, Sigma-Aldrich) was dissolved in acetone at the final concentration of 2 mg/mL. An amount of 5×10¹⁰ EVs/mL was incubated with FDA (18 μmol) in 0.2 μm-filtered PBS (without Ca⁺⁺ and Mg⁺⁺), in a final volume of 200 μl. As a negative control, the same amount of FDA was incubated in PBS (without Ca⁺⁺ and Mg⁺⁺, 0.2 μm-filtered). The fluorescence emission was followed for up to 20 hours at 22° C. (room temperature), using the spectrofluorimeter (ex/em 490/514 nm, GloMax Discover Microplate Reader, Promega).

### Measurement of the Enzymatic Activity

To calculate the specific enzymatic activity, the enzyme unit (U) was determined, i.e., the amount of enzyme that catalyses the transformation of 1 nanomole (nmol) of product (fluorescein) per minute. Therefore, the fluorescence intensity was interpolated on the fluorescein calibration curve. This value of the amount of fluorescein was divided by the assay duration time, expressed in minutes. The amount of fluorescein produced per unit of time during the method was thereby obtained, resulting in a value which is an index of the enzymatic activity of the vesicles expressed in enzymatic units, i.e., as nanomoles of fluorescein produced per minute (nmol/min).

### Michaelis-Menten Study

An amount of 5×10¹⁰ EVs/mL was incubated with different concentrations of FDA (0, 1.6, 3.3, 6.6, 13, 26×10³ nmol) in 0.2 μm-filtered PBS (without Ca⁺⁺ and Mg⁺⁺), in a final volume of 200 μl. The fluorescence emission was followed for up to 3 hours (180 minutes) using the spectrofluorimeter. Deriving the nanomoles (nmol) of fluorescein produced via the calibration curve, the Michaelis-Menten equation was applied using a nonlinear regression (FIG. 3). This study allowed the Vmax and Km of the enzymes in the vesicles to be obtained.

### Analytical Specificity

In order to verify the ability of the method according to the invention to discriminate between intact and damaged vesicles, 5×10¹⁰ EVs/mL were incubated with 1% Triton X-100 (Sigma-Aldrich) for 30 minutes at room temperature. The same amount of vesicles was subjected to boiling at 100° C. for 10 minutes.

Subsequently, the samples were incubated with 18 μmol FDA, reaching the final volume of 200 μl with PBS (without Ca⁺⁺ and Mg⁺⁺, 0.2 μm-filtered). The fluorescence emission was followed for up to 3 hours (180 minutes) and the enzymatic activity was determined as described above. As a negative control, the same amount of FDA was incubated in PBS (without Ca++ and Mg++, 0.2 μm-filtered), subjected to boiling at 100° C. for 10 minutes or with 1% Triton X-100.

### Batch-to-Batch Reproducibility of Extracellular Vesicle Preparations

An amount of 5×10¹⁰ EVs/mL from different batches (designated as N1, N2 and N3) was incubated with 18 μmol FDA, reaching the final volume of 200 μl with PBS. The fluorescence emission was followed for up to 3 hours (180 minutes) and the enzymatic activity was determined as described above. As a negative control, the same batches of vesicle preparations were subjected to boiling at 100° C. for 10 minutes.

### Assessment of the Quality of EV Preparations Following Different Storage Conditions

The vesicles were stored for 10 days under different conditions: 4° C., −20° C., −80° C. and after freeze drying in 5% and 8% sucrose. The freeze-dried samples were thoroughly rehydrated in 0.2 μm-filtered Milli-Q water and resuspended to ensure complete dispersion. After performing the nanoparticle tracking analysis (NTA) on all samples, the enzymatic assay was carried out, as previously described.

### Assessment of the Quality of EV Preparations Following Different Treatments for Vesicle Loading

An amount of 2×10¹² vesicles (and 0.2 μm-filtered PBS used as a negative control) were subjected to several physical treatments already described in the literature as physical methods for loading exogenous molecules into vesicles, including electroporation, sonication, freeze-thaw, saponification, and extrusion.

In short, the electroporation was performed in Gene Pulser cuvettes (0.4 cm) on a Gene Pulser BioRad equipped with a capacitance extender, with two selected conditions: E1 (125 μF, 400 V and two 20-ms pulses) and E2 (125 μF, 250 V and two 30-ms pulses). The sonication was performed using two settings: sonicator (S1) at 20% amplitude for six 30-second on/off cycles for a total of 3 minutes, with 2 minutes of cooling, then incubation for 60 minutes at 37° C.; 40 KHz ultrasonic bath (S2), at 40% amplitude for two 30-second on/off cycles, then incubation for 60 minutes at 37° C. The freeze-thaw was performed with 3 cycles of freezing at −80° for 30 minutes and thawing at room temperature for 30 minutes. The saponification was performed using two settings: incubation with 0.1 mg/ml (Sap1) and 0.002 mg/ml (Sap2) saponin (Sigma-Aldrich) at room temperature for 10 minutes. The extrusion was carried out using a manual syringe mini-extruder fitted with polycarbonate membranes with pore sizes of 100 nm and 200 nm (Tracketch membrane, Whatman). Each sample was extruded 31 times.

After each method was run, all samples were subjected to NTA. Subsequently, the functionality test of the invention was conducted, as previously described.

Evaluation of Sample Quality after Different Isolation Methods

An amount of 5×10¹⁰ EVs/mL, coming from the same batch but isolated by differential ultracentrifugation and tangential flow filtration, was incubated with 18 μmol FDA, reaching the final volume of 200 μl with 0.2 μm-filtered PBS.

The fluorescence emission was followed for up to 3 hours (180 minutes) and the enzymatic activity was determined.

### Application of the Method of the Invention to Preparations of Extracellular Vesicles of Human Origin

An amount of 5×10¹⁰ EVs/mL isolated from tumour and non-tumour mammary cells (MDA MB231, 1-7 HB2) and human embryonic kidney cells (HEK 293T) were incubated with 18 μmol FDA, reaching the final volume of 200 μl. The fluorescence emission was followed for up to 3 hours (180 minutes) and the enzymatic activity was determined.

### Statistical Analysis

Each measurement was repeated in triplicate and the mean values and standard deviations were calculated. One-way ANOVA analysis was performed with GraphPad software.

### Results

### Example 1: Proof of Concept of the Method According to the Invention

In carrying out a proof of concept of the method according to the invention, the present inventors initially established that said method was capable of providing a universally quantifiable and standardizable final value.

For this purpose, the temperature was defined (room temperature, approximately 22° C.) as well as the duration of the assay, setting it equal to 3 hours (180 minutes, i.e., a time when the fluorescence emission intensity from FDA hydrolysis is in the exponential phase of the reaction, far from the plateau), in order to make the assay fast, sufficiently sensitive, and easy to perform.

(Nano)algosomes, i.e., EVs isolated from microalgae, were used as model vesicles. In order to determine the amount of FDA enzymatically cleaved during the assay to form fluorescein, thus making the assay quantitative, a standard calibration curve was created using fluorescein (FIG. 2). The calibration curve obtained had a linear pattern, which allowed the intensity of the fluorescence emitted following the hydrolysis of the FDA substrate to be converted into an amount (nmol) of fluorescein produced, for each EV preparation sample tested.

By dividing the amount of fluorescein produced by the total time established for the assay, i.e., 180 minutes (3 hours), the amount of fluorescein produced per unit of time was obtained (nanomoles/minute), which corresponds to the specific enzymatic activity units (U=nmol/min).

To establish the optimal conditions for an enzymatic reaction, the inventors applied the Michaelis-Menten model, one of the best-known and most useful approaches to study enzyme kinetics (FIG. 3). By carrying out the method of the invention in the presence of different concentrations of the FDA substrate and by measuring the initial rate of the reaction at constant concentrations of vesicles, a hyperbolic relationship was obtained. By applying a nonlinear regression to the Michaelis-Menten equation, key kinetic parameters, such as the maximum velocity and the Michaelis-Menten constant (Vmax and Km), were obtained.

In particular, a maximum reaction velocity of 2.9 nmol/min was detected and a Km of 3 μmol was measured. These values demonstrate a high affinity of enzymes present in the extracellular vesicles of microalgae for the FDA substrate.

Since the concentration of the substrate to be used in a reaction must be higher than the Km value, the concentration of the substrate (FDA) was set at a value 6 times higher than the Km, i.e., 18 μmol.

### Example 2: Analytical Sensitivity of the Method of the Invention

In order to assess the analytical sensitivity of the method according to the invention, the present inventors determined the lower limit of detection of the functionality assay by identifying the lowest amount of EVs that can be distinguished from the background.

For this purpose, several amounts of (nano)algosomes (1×10¹¹ to 6×10⁸ EVs/mL) from the same batch of a preparation of EVs were incubated with FDA, and with the method according to the invention a dose-response relationship was observed with a limit of detection higher than 6.25×10⁹ EVs/mL (FIG. 4).

### Example 3: Validation of the Method According to the Invention

In order to confirm that the method according to the invention depends on the activity of intravesicular enzymes and requires the integrity of the EVs, a non-ionic detergent such as TritonX100 was used, which makes the lipid membranes permeable but does not affect the activity of proteins, including enzymes.

The results of the assay of the invention illustrated in FIG. 5 showed that the production of fluorescein from FDA depends on active enzymes present in intact vesicles. In fact, when the membrane of the vesicles is damaged, there is no fluorescein build-up therein, so no fluorescence value can be obtained that is measurable or distinguishable from the background.

Otherwise, the retention of fluorescein within vesicles not treated with the detergent yields a quantifiable value that is related to the enzymatic activity of intact EVs, thus indicating that the method of the invention is suitable for detecting the enzymatic activity of intact EVs.

The same results were obtained when the EVs in the preparations were subjected to lysis by boiling at 100° C. for 10 minutes, confirming the specificity of the method of the invention (FIG. 5). In the experiments described above, the measurements were performed with three technical replicates, confirming the high reproducibility.

### Example 4: Evaluation of the Quality of EV Preparations

Evaluating the quality of a preparation of extracellular vesicles is a key requirement to improve experimental reproducibility during clinical and therapeutic applications.

As described in the publications of Adamo G. et al, J. Extracellular Vesicles, 2021, 10, e12081, and Paterna A. et al 2021, Front Bioeng Biotechnol. 2022; 10:836747, the strategy of producing EVs from microalgae was optimized by evaluating quality parameters suggested by the MISEV2018 guidelines, including extensive biophysical and biochemical characterization using different techniques, such as Dynamic Light Scattering (DLS), the dispersion and fluorescence Nanoparticle Tracking Analysis technology (NTA and F-NTA), protein analysis (BCA), western blot analysis, and Atomic Force Microscopy (AFM). The implementation of these quality controls therefore allowed large-scale TFF isolation of microalgal EVs to be optimized.

The application of the method of the invention allowed different vesicle productions to be monitored in terms of sample quality. These differences are mainly due to the performance of the cartridges used during vesicle isolation by TFF.

In this context, the inventors selected several batches of vesicles with differences in quality (N1, N2, N3), assessed on the basis of the unbalanced relationship between the amount (micrograms) of proteins present in the EVs and the concentration in terms of number of particles (measured by NTA). In addition, the concentration of the different batches was determined with the F-NTA technique using fluorescent staining with Di-8-ANEPPS, a fluorescent EV-specific lipophilic dye that assists the discrimination of contaminants in vesicle preparations. This analysis showed that the selected EV batches actually contained several contaminants or co-isolates.

The results obtained with the method of the invention on the aforementioned preparations showed that the enzymatic activity measured in the different batches of vesicle preparations was completely consistent with the results of the F-NTA, NTA and protein analyses (FIG. 6). This result confirms the method according to the invention as a new method that allows, in a single step, EV preparations to be validated and batch-to-batch variability during vesicle production to be monitored.

It should also be noted that the method of the invention does not require any washing step, thus offering several advantages: simplicity, cost-effectiveness, sensitivity, and speed.

### Example 5: Application of the Method According to the Invention

### Evaluation of the Conditions of Storage of EV Preparations

Numerous studies have shown that several factors related to the conditions of storage of EV preparations can affect various characteristics of the vesicles, including membrane stability and EV function. Recently, a study systematically compared different EV cryopreservation strategies in order to identify the most appropriate conditions to stabilize extracellular vesicle preparations over time, especially for therapeutic applications, such as the development of new vaccines.

Typically, after storage, EV preparations are checked with various methods, including the NTA technique or flow cytometry (IFCM). In this context, the present inventors conducted dedicated experiments using the method according to the invention to compare the effect of different storage conditions on EV preparations, including temperatures at 4° C., −20° C., −80° C., or freeze drying in 5% or 8% sucrose. The size and numerical concentration of vesicles stored under these different conditions were also monitored by NTA, but no significant difference in EV concentration was observed for the conditions tested.

As shown in FIG. 7, the method of the invention rapidly detects a significant and quantifiable reduction in EV functionality, which results in reduced enzymatic activity in EV samples that have been frozen and thawed (at −20° C. or −80° C.) or freeze-dried and resuspended compared to vesicle preparations stored at 4° C. for one week.

### Evaluation of Methods of Loading Extracellular Vesicles

As is known, EVs represent an advantageous drug delivery system due to their low toxicity, high targeting capacity and slow clearance. The exogenous load can be loaded into the EVs using various physical methods, including electroporation, sonication, saponin-assisted loading, freeze and thaw cycles, and extrusion. However, these methods can cause EVs to lose function or damage the integrity of their membranes, even if the phospholipid bilayer rapidly recovers its integrity after membrane perturbations.

In this context, the inventors therefore verified the possible application of the functionality assay of the invention in order to identify the loading method that has the least impact on the integrity and bio-activity of the EVs.

For this evaluation, several EV loading methods were used (FIG. 8). The results obtained with the assay of the invention showed that freezing and thawing is the “gentlest” method of loading the vesicles, exhibiting the least impact on the characteristics of the EVs. In contrast, for the other conditions (electroporation or sonication) a decrease in the enzymatic activity of the EVs was obtained, probably due to severe membrane disturbance or loss of vesicle integrity.

The application of the method according to the invention for the evaluation of the functionality of extracellular vesicles in relation to the loading strategy is an important starting point for the development of therapies based on the use of EVs.

### Evaluation of Methods of Isolating Extracellular Vesicles

Various approaches are employed in the art to isolate extracellular vesicles derived from different biological sources. Based on the specific experimental settings (such as, for example, small- or large-scale production), it is essential to determine the most efficient method for separating EVs, also taking into account the quality of the preparations thus obtained. The studies conducted by Adamo et al. 2021 et al, J. Extracellular Vesicles, 2021, 10, e12081 demonstrated that it is possible to isolate (nano)algosomes from Tetraselmis chuii conditioned medium by using different separation procedures (e.g., differential ultracentrifugation or dUC, tangential flow filtration or TFF, and density gradient ultracentrifugation or gUC).

During the validation of the performance of the method of the invention, the inventors therefore used the functionality assay according to the invention to compare the quality of (nano)algosome preparations deriving from the same culture batch as Tetrasemis chuii, isolated in parallel by differential ultracentrifugation and tangential filtration. As shown in the graph in FIG. 9, comparable measures of functionality (bioactivity and membrane integrity) were observed in the vesicle preparations obtained with the above methods, thus confirming the good quality of these preparations.

The above results therefore indicate that the method of the invention allows a direct and quantitative comparison of the quality of the EV preparations in relation to the methods used for isolating the vesicles.

### Analysis of Preparations of Extracellular Vesicles of Human Origin

The characterization of preparations of extracellular vesicles isolated from human cells or biological fluids is critical for the diagnostic and therapeutic applications of EVs. Versatile and robust characterization methods for higher precision, with higher sensitivity, starting from a smaller amount of EVs are therefore needed.

In order to verify whether the method of the present invention satisfies these characteristics, the inventors carried out experiments to assess whether said method is sufficiently sensitive, compatible, and specific for determining the functionality of preparations of EVs isolated from human cells. In short, vesicles were isolated from several human cell lines, namely normal and tumour mammary epithelial cells and human embryonic kidney cells. After the biochemical/biophysical characterization of the three different types of EVs, the preparations were incubated with FDA to perform the functionality assay of the invention. The results obtained demonstrate the presence of a reliable enzyme-dependent activity for the three types of EVs analysed (FIG. 9), confirming the compatibility of the method of the invention also for direct applications on preparations of EVs of human origin. Notably, a significantly higher enzymatic activity was observed in extracellular vesicles derived from breast cancer cells compared to EVs isolated from normal cells of the same tissue, opening up new perspectives and applications of the functionality assay of the invention for the diagnosis of tumours.

On the basis of the results of the validation study carried out by the present inventors, it is therefore apparent that the method according to the invention represents a new method that can be widely used in the field of extracellular vesicles, with countless applications, allowing in fact a general evaluation of the functionality of EV preparations of different types and representative of different pathological conditions. Such a method can also anticipate the results of specific potency assays; indeed, the result of the method of the invention showing a high functionality of EV preparations may be the essential prerequisite for subsequent therapeutic use and therefore for the related potency assay.

What is claimed is:

1. A method of evaluating the functionality of a preparation of extracellular vesicles (EVs), the method comprising steps of:
(i) providing a preparation of EVs;
(ii) incubating the preparation of EVs with fluorescein diacetate (FDA) as a non-fluorescent precursor of fluorescein under suitable conditions so that the FDA enters into the EVs of the preparation and undergoes intravesicular hydrolysis by esterase enzymes to form fluorescence-emitting fluorescein;
(iii) detecting by spectrofluorometry an intensity of the fluorescence emitted over time at a wavelength of 516 nm and at a temperature between 21° C. and 25° C., and measuring an intensity value of the fluorescence emitted at a time t of the hydrolysis reaction (Iₜ);
(iv) determining from the Iₜ value measured in step (iii) an amount of fluorescein produced at the time t in the preparation of EVs (Fₜ), based on a calibration curve relating to the intensity of the fluorescence emitted at the wavelength of 516 nm as a function of the amount of fluorescein; and
(v) calculating the amount of fluorescein produced per unit of time in the preparation of EVs (F) by applying the formula:
$$F = \frac{Ft}{t}$$
wherein the amount of fluorescein produced per unit of time in the preparation of EVs is correlated to the functionality of the preparation of EVs.

2. The method of claim 1, wherein the FDA is added to the preparation of EVs at a concentration comprised within a range of 0.0005% to 0.001% w/v based on a total volume of the preparation of EVs.

3. The method of claim 1, wherein in step (iii) the temperature is 22° C.

4. The method of claim 1, wherein the time t of the hydrolysis reaction is comprised within a range of 60 to 600 minutes.

5. The method of claim 4, wherein the time t of the hydrolysis reaction is 180 minutes.

6. The method of claim 1, wherein the EVs are derived from a biological fluid or from a conditioned cell or tissue culture medium.

7. The method of claim 6, wherein the biological fluid is blood, saliva, plasma, serum, urine or cerebrospinal fluid.

8. The method of claim 1, wherein the EVs are derived from cells selected from the group consisting of normal and cancerous animal cells, animal stem cells, normal plant cells, plant stem cells, bacteria, microalgae, and any combination thereof.

9. The method of claim 1, wherein the fluorescein of the calibration curve is fluorescein sodium salt.
